(19) United States
(12) Patent Application Publication
(10) Pub. No.: US 2005/0208034 A1
(43) Pub. Date: Sep. 22, 2005

(54) Natural vaginal yeast infection control

(76) Inventor: Lazonia Victoria Jordan, Duluth, GA (US)

Correspondence Address: Lazonia Victoria Jordan;SpectaCure, Inc., Suite 5396A, 2180 Pleasant Hill Road, Duluth, GA, 30096, US

(21) Appl. No.: 10/801,297

(22) Filed: Mar. 17, 2004

(51) Int. Cl. A61K 45/00; A61K 35/78

(52) U.S. Cl. 424/93.45; 424/754; 424/769

(57) ABSTRACT

A nonprescription natural source to curing, preventing, controlling and managing vaginal yeast infections. This natural solution is available in tablet, capsule or powder formulation comprised of pure, natural, safe ingredients. This invention is ingested orally.

### CROSS-REFERENCE TO RELATED APPLICATIONS

Not Applicable

### FEDERALLY SPONSORED RESEARCH OR DEVELOPMENT

There are no rights to this invention under federally sponsored research and development.

### SEQUENCE LISTING, A TABLE OR A COMPUTER PROGRAM LISTING COMPACT DISK APPENDIX

Not Applicable

### BACKGROUND OF THE INVENTION

1. Field of Invention

This invention relates to feminine vaginal yeast infections, specifically to a natural remedy to cure, control and prevent vaginal yeast infections.

2. Discussion of Prior Art

Because of hormonal changes and imbalances in the pH of the vaginal lining, over 7 million women across the world experience monthly recurring yeast infections. Currently, the only solutions to the aggravating symptoms women experience due to the overgrowth of candida yeast are messy topical vaginal creams, suppositories or vaginal inserts which are not always effective, and the Diflucan pill which is taken orally, and is also often an ineffective treatment. The vaginal inserts, creams as well as the prescription strength Diflucan pill, on many occasions do not cure the infections causing women to have to spend even more monies to purchase even more expensive prescription creams; thus expending monthly to get the yeast problem under control. In addition, to often being ineffective, the Diflucan pill is a chemically compound drug, which frequently causes painful side affects for some women. During the treatment with vaginal creams or suppositories and the Diflucan pill which are now available to treat vaginal yeast infections, a woman has to wait days before relief is gained; thus causing continued itching and burning even after beginning treatment with one of the current yeast infection treatments. Additionally, treatments which are currently on the market to treat vaginal yeast infections are made with chemicals and do not address the core of the problem which causes the infections. For this reason, 90% of the women who experience yeast infections experience them monthly going through a vicious cycle of multiple uses of ineffective drugs before experiencing relief before the next month's occurrence. Some vaginal yeast infection treatments which are a bit more effective than over-the-counter treatments are available only by prescription which can cause such inconveniences as getting a physician to write a prescription and sometimes an unnecessary expensive doctor's office visit.

Common causes of vaginal yeast infections are pregnancy, menstruation, taking birth control pills and the use of antibiotics. More common than others simply menstruating causes many women to experience yeast infections. Using the creams and suppositories during this time is an extremely gruesome unpleasant task.

Objects and Advantages

Several objects and advantages of my invention are that this invention offers women a clean, natural and effective treatment of vaginal yeast infections. This invention targets the hormonal imbalances, which causes women to experience yeast infections thus eliminating or minimizing the discomforts women experience from monthly and occasional occurrences of yeast infections. In fact, in many of the cases treated with this invention, women no longer experience vaginal yeast infections and if they do, relief from the infection with the treatment of this invention is experienced within minutes of the intake of this invention. This invention also eliminates the infections women experience without multiple treatments for the same infection. This invention gives women the ability to can eat, drink and participate in any physical activities that normally would cause yeast infections to occur. Since this invention is a natural cure, no prescription is required for its use; hence no prescriptions and no doctor's visits.

Due to its being a natural solution, this invention can also be taken concurrent with the antibiotics which may be being taken for the treatment of other infectious diseases. This invention, being made of natural ingredients, also lends a viable solution to the discomforts experienced due to vaginal yeast infections to pregnant women.

A common time that a woman needs to be treated for a yeast infection is during menstruation. This solution offers an extremely clean method for the treatment of a yeast infection during that delicate time of the month. It also eliminates a woman being inconvenienced with administering vaginal creams during her menstruation cycle.

Because of the composition of natural ingredients for this invention, it can be administered to individuals who are taking other medications for other illnesses.

This invention provides women with a natural, safe, simple, clean, reliable and effective alternative to the management of vaginal yeast infections. Further, from a consideration of the ensuing description of my invention, objects and advantages of my invention will become apparent.

### SUMMARY OF THE INVENTION

Said invention is comprised of a combination of natural ingredients to cure and control the overgrowth of undesirable candida yeast in the female's vaginal tract. This solution is clean, neat and causes no dangerous side affects because its ingredients are all natural. This invention may have other healthful benefits such as the maintaining cholesterol within normal range, supporting heart and cardiovascular health, aiding in maintaining health intestinal tracts and providing immune system support. This invention is available in pill or capsule form.

### DRAWINGS

Not Applicable

### DETAILED DESCRIPTION OF THE INVENTION

The natural ingredients combined in tablet, capsule or powder forms when taken orally prevents, cures and controls the occurrence of uncomfortable, irritating vaginal yeast infections. Vaginal candida yeast infections occur due to women's natural hormone changes, the intake of certain foods and drinks, the wearing of certain clothing, perspiration during exercise and many other things. With this new invention, a woman can eat or wear whatever she desires. She does not have to worry about many of the uncontrollable monthly hormone and pH imbalances. By orally taking SpectaCure's Natural Feminine Vaginal Yeast Control tablet, capsule or powder formulation, a woman can wear, eat or drink whatever she desires without the worries of getting a vaginal yeast infection. The tablet, capsule or powder formulation can be taken with or after eating the foods or drinks that promote yeast infections. If a woman wears clothing and begins to feel the onset of a yeast infection because of that attire, she can take a tablet, capsule or powder formulation to relieve the symptoms. The tablet, capsule or powder formulation may be taken even after the onset of an infection with excellent and successful results. Relief is experienced within 5 to 10 minutes of ingestion of the formulation.

The main active ingredients for the tablets, capsules and powder formulations are as follows:

Pau D' Arco or La Pacho (tabebuia heptaphylla, inner bark)-500 mg
Acidophilus—18 billion organisms per gram (lactobacillus acidophilus—2 billion, bifidobacterium bifidum—1.2 billion, bifidobacterium longum—200 million, streptococcus thermophilus—200 million, lactobacillus bulgaricus—200 million, lactobacillus paracasei—200 million)
Calcium as dicalcium Phosphate—56 mg
Allium sativum or concentrated Garlic bulb—500 mg
   Other ingredients:
Cellulose (plant fiber)
Magnesium Stearate
Silica

### CONCLUSION, RAMIFICATIONS AND SCOPE OF INVENTION

Thus it can be seen that the Natural Vaginal Yeast Infection Control can be used by women of all ages who suffer from the complications of vaginal yeast infections caused by the over growth of candida yeast. It is a natural, safe, simple, clean, reliable and effective treatment for curing, controlling and managing vaginal yeast infections.

While there are many specificities pertaining to my invention in the above description, these specificities should not be construed as limitations on the scope of the invention, but rather as an exemplification of one preferred embodiment thereof. Other variations are possible. For example, instead of combining all three active ingredients just the garlic and pau d' arco can be formed in a single pill to achieve similar results. The pills can be made in smaller sizes for easier intake. They can be shaped round, square or oval shaped caplets. Accordingly, the scope of the invention should be determined not by the embodiment described herein, but by the appended claims and their legal equivalents.

1. Tablets, capsules and powder formulations developed with natural ingredients taken orally to prevent, cure, control and/or manage vaginal yeast infections.

2. A method as stated in claim 1 wherein the natural ingredients for the tablets, capsules and powder formulations are comprised and compounded of the active natural ingredients Pau D' Arco or La Pacho (tabebuia heptaphylla, inner bark)-500 mg, Acidophilus—18 billion organisms per gram (lactobacillus acidophilus—2 billion, bifidobacterium bifidum—1.2 billion, bifidobacterium longum—200 million, streptococcus thermophilus—200 million, lactobacillus bulgaricus—200 million, lactobacillus paracasei—200 million) and Allium sativum or concentrated Garlic bulb—500 mg.

3. A method as recited in claim 1 comprising the further step of oral administration of the tablets, capsules or powder formulations to manage and control the pH balance of the vaginal tract thus preventing, curing and controlling yeast infections in the vaginal tract of the vaginal tract.

4. A method as recited in claim 1 wherein the effective dosage is 1 to 3 capsules or tablets per day or a measurement of powder to the equivalent of the ingredients outline in claim 2 depending on the severity of the yeast infection being treated.

5. A method as recited in claim 2 wherein the dosage is administered with each meal.

6. A method as recited in claim 2 wherein the Acidophilus may be eliminated from the compound.

7. A method as recited in claim 2 wherein said preparation includes the following inactive ingredients: Cellulose (plant fiber), Magnesium Stearate and Silica.

8. A method as recited in claim 1 wherein the composition is administered at least three times a day for at least two weeks upon the initial treatment with the invention, dosage outlined in claim 2.

9. A method as recited in claim 1 wherein the composition is administered on an as needed basis.

10. A method as recited in claim 1 wherein the powder formulation may be administered by mixing the powder in any beverage for consumption.

11. A method as recited in claim 1 wherein individuals who are being treated with other drugs for other illnesses may ingest the tablet, the capsule or the powder formulation with no complications due to the interaction of this naturally formulated invention with the other drugs.
